(19) United States
(12) Patent Application Publication
(10) Pub. No.: US 2015/0290018 A1
(43) Pub. Date: Oct. 15, 2015

(54) SUPPORTER

(71) Applicant: MORITO CO., LTD., Osaka (JP)

(72) Inventors: Hisashi Okazawa, Osaka-shi (JP); Hajime Setoguchi, Osaka-shi (JP)

(73) Assignee: MORITO CO., LTD., Osaka (JP)

(21) Appl. No.: 14/438,649

(22) PCT Filed: Oct. 10, 2013
(86) PCT No.: PCT/JP2013/077573

§ 371 Date: Apr. 27, 2015

(30) Foreign Application Priority Data
Oct. 26, 2012 (JP) 2012-236546
May 27, 2013 (JP) 2013-110566

(51) Int. Cl. A61F 5/02; A41D 19/00; A41B 11/02

(52) U.S. Cl. CPC A61F 5/028; A41B 11/02; A41D 19/00

(57) ABSTRACT

Provided is a supporter that has an appropriate bending property and is inhibited from excessively bending A supporter (4) includes a base (41), a plurality of grooves (42) that are provided in the width direction of the base (41), fillers (43) that can be respectively inserted into the grooves (42), and disconnection preventing lips (44) for the fillers (43). The fillers (43) each have a small protrusion (49) that extends downward, the base (41) has a through hole (45) or a depression in each of the grooves (42) , and the small protrusion (49) and the through hole (45) can be fitted to each other and freely move to such an extent that they do not separate from each other.

### TECHNICAL FIELD

The present invention relates to a supporter for protecting or mending the neck, a wrist, an ankle, the lumbar region, a joint, and the like of a human body by being placed thereon. Specific examples include the following items.

(1) A supporter for a neck guard that is a part of a strap that is to be used by being worn on the neck in order to support a musical instrument, a camera, binoculars, and the like, and that is to be used for distributing load on the neck

(2) A supporter for a wrist guard for preventing injuries that may be caused due to a wrist being bent backwards abnormally in various types of sports or from a sudden action.

(3) A supporter for an ankle guard for preventing injuries that may be caused due to an ankle being bent abnormally in various types of sports or from a sudden action.

(4) A supporter for a joint protector that has a warping preventing lip, suppresses a large movement of a joint that is excessive, and assists the improvement of competitive ability in various types of sports.

(5) A supporter for a lumbar region belt that is to be used for prevention and treatment of lumbar pain.

### BACKGROUND ART

Various types of supporters such as a medical splint and an elastic cloth for covering have been known. The medical splint is used for immobilizing an affected area, and thus usually it is assumed that the medical splint doesn't move. With the elastic cloth for covering, it is not possible to prevent a specific area of a human body from bending abnormally in various types of sports or from a sudden action.

In reality, there are cases in which complete immobilization is not desired, but easy displacement is not favorable. There is a demand for a supporter that is moderately immobilized and has a degree of freedom to some extent

For example, because a musical instrument such as a saxophone or a guitar is quite heavy, in order to lessen a burden, the musical instrument is connected to a strap and the strap is worn on the neck of a player when playing the musical instrument. However, if the player plays the musical instrument for a long period of time, a string-like strap is likely to dig into the neck and cause a pain in the neck Accordingly, wearing such a strap often results in problems such as difficulties in breathing while the player is playing the musical instrument

In order to improve this, inventions and ideas in which the strap is provided with a neck guard and lies on the neck in a planar manner instead of a linear manner are known. Examples thereof include Japanese Patent No. 4296228 (Patent Document 1), JP 2008-268737A (Patent Document 2), and the like.

It is important for this type of neck guard to distribute the load evenly so pressure is acceptable. In order to achieve this, a neck guard whose flexibility is too small or too large is not acceptable. If the flexibility is significantly small, the contact area with the neck is reduced, and thus the load is likely to be linear, whereas if the flexibility is significantly large, the neck guard is excessively bent, and thus the load is not distributed even if the contact area with the neck increases.

In other words, there is a demand for a supporter that can be moderately immobilized and has a degree of freedom to some extent

There is a similar demand for a wrist supporter, ankle supporter, lumbar region supporter, and joint supporter.

### CITATION LIST

### Patent Document

Patent Document 1: Japanese Patent No. 4296228
Patent Document 2: JP 2008-268737A

### SUMMARY OF THE INVENTION

### Problem to be Solved by the Invention

The present invention has been made in view of issues of the above-described conventional techniques, and an object of the present invention is to provide a supporter that has a function of providing appropriate flexibility and of suppressing excessive bending.

### Means for Solving the Problem

The present invention is a supporter for a human body, and the supporter includes a base, a plurality of grooves that are provided in a width direction of the base, fillers that can be respectively inserted into the grooves, and disconnection preventing lips for the fillers.

Preferably, the fillers each have a small protrusion that extends downward, the base has a through hole or a depression in each of the grooves, and the small protrusion and the through hole can be fitted to each other and freely move to such an extent that they do not separate from each other.

Preferably, the disconnection preventing lips are extending portions that each extend from two sides of an upper end of a groove toward the groove.

Preferably, the fillers each have a shape complementary to the grooves and can be inserted therein in a sliding manner in the lateral direction.

The supporter of the present invention can be used as a neck supporter, a wrist supporter, an ankle supporter, a lumbar region supporter, and a joint supporter.

### Effects of the Invention

According to the present invention, this supporter has gaps between the base and the fillers when in a natural state and thus can be bent. However, if the supporter is excessively bent, the gaps are closed, and if the supporter is excessively bent beyond a fixed range, the supporter is completely inhibited from bending, and thus an appropriate pressure is applied.

With a preferable embodiment, a small protrusion and a through hole are not closely fixed to each other using an adhesive or the like, and freely move to some extent while maintaining a fitted state without being separated from each other. A base portion and the fillers are not fixed, and thereby it is possible to get an advantage in that the maximum flexibility during bending (the supporter bends in a natural curve) is achieved. Also, because the supporter is not bent in a predetermined curvature or greater, excessive bending can be suppressed.

Hereinafter, examples of the present invention will be described with reference to accompanying drawings.

### BRIEF DESCRIPTION OF THE DRAWINGS

FIG. 1 is a perspective view of a supporter according to an example of the present invention in a non-assembled state.

FIG. 2 is a perspective view of the supporter of FIG. 1 in an assembled state.

FIG. 3(a) is a cross-sectional view of the supporter of FIG. 1 in a normal state, and FIG. 3(b) is a cross-sectional view thereof when load is applied thereto.

FIGS. 4(a) and 4(b) are respective enlarged views of circles (a) and (b) shown in FIG. 3.

FIG. 5 is a perspective view of a strap having a supporter for a neck guard, which is a first use example of the present invention.

FIG. 6 is (a) a top view, (b) a bottom view, (c) a cross-sectional view, and (d) a side view of the neck guard of FIG. 5.

FIG. 7 is (a) a rear view and (b) a top view of a lumbar region belt having a lumbar region supporter, which is a second use example of the present invention.

FIG. 8 is a perspective view of a glove having a wrist supporter, which is a third use example of the present invention.

### DESCRIPTION OF EMBODIMENTS

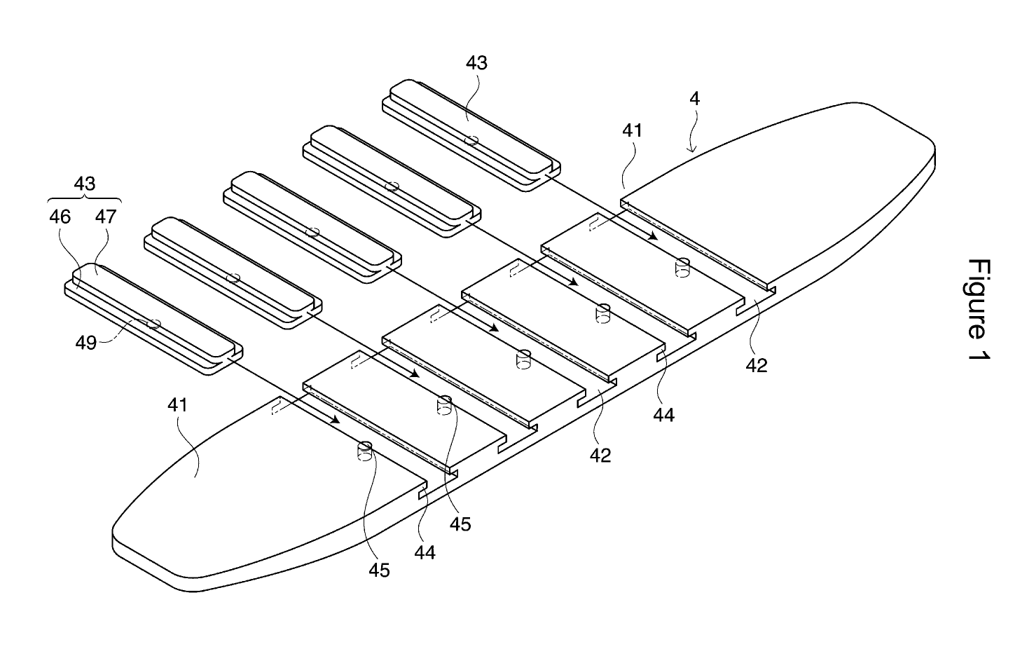

As shown in FIGS. 1 to 4, a supporter 4 includes a base 41 that is a synthetic resin plate having an elongated ellipse shape (18.5 cm×4 cm) and that has a plurality of grooves 42 (five in this example) on the surface, and strip-like fillers 43 that can be respectively inserted into the grooves 42. The supporter 4 can be formed by injection molding using a synthetic resin such as nylon that is firm and has elasticity.

Each groove 42 has a width of approximately 1 cm and the bottom of the groove 42 is formed flat. A disconnection preventing lip 44 is provided so that the inserted filler 43 doesn't disconnect from the groove. The disconnection preventing lip 44 can be an extending portion that extends from two sides of the upper end of the groove 42 toward the groove by approximately 2 mm.

One through hole 45 is provided at the center in the longitudinal direction and the width direction of each groove.

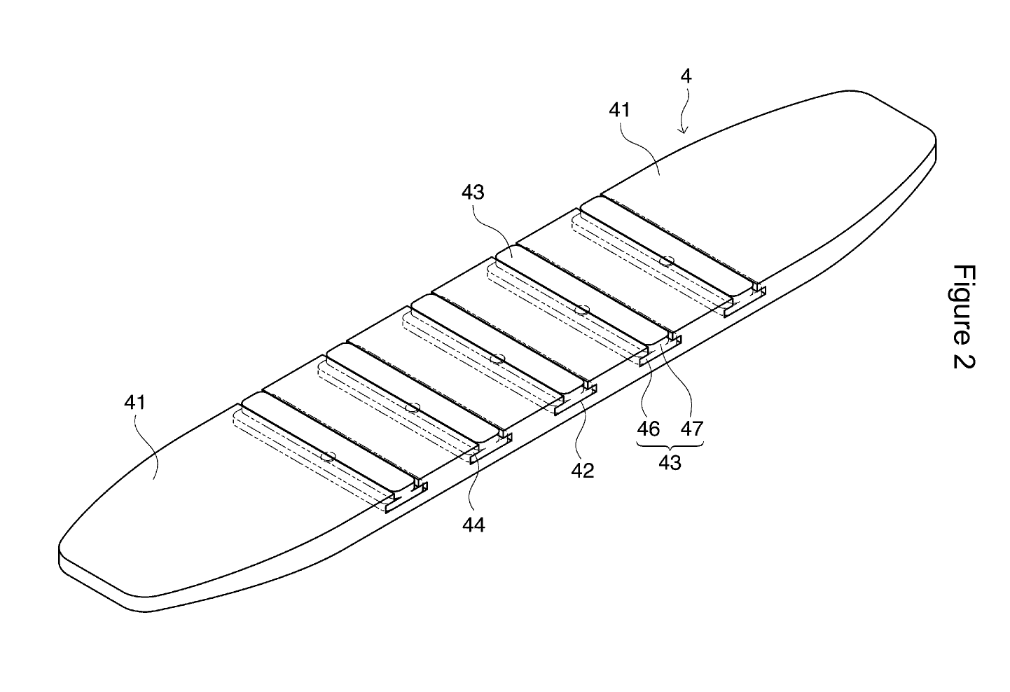

The filler 43 has a cross-sectional shape complementary to the groove 42. In other words, the filler 43 has two layers, namely a base portion 46 corresponding to the groove width of the supporter 4, and a narrow-width portion 47 due to the disconnection preventing lip. Therefore, if the filler is inserted in a sliding manner into the groove in the direction of arrows shown in FIG. 1, it closely fits into the groove as shown in FIG. 2, and the height of the top surface thereof is the same as the supporter.

The base portion 46 has a small protrusion 49 that has a height of approximately 0.5 to 1 mm and extends downward, and the small protrusion 49 is fitted into the through hole 45 of the groove 42. The small protrusion 49 and the through hole 45 are not closely fixed using an adhesive or the like, and freely move to some extent while maintaining a fitted state without being separated from each other.

The base portion 46 and the fillers 43 are not fixed, and thereby it is possible to get an advantage in that the maximum flexibility during bending (the supporter bends in a natural curve) is achieved.

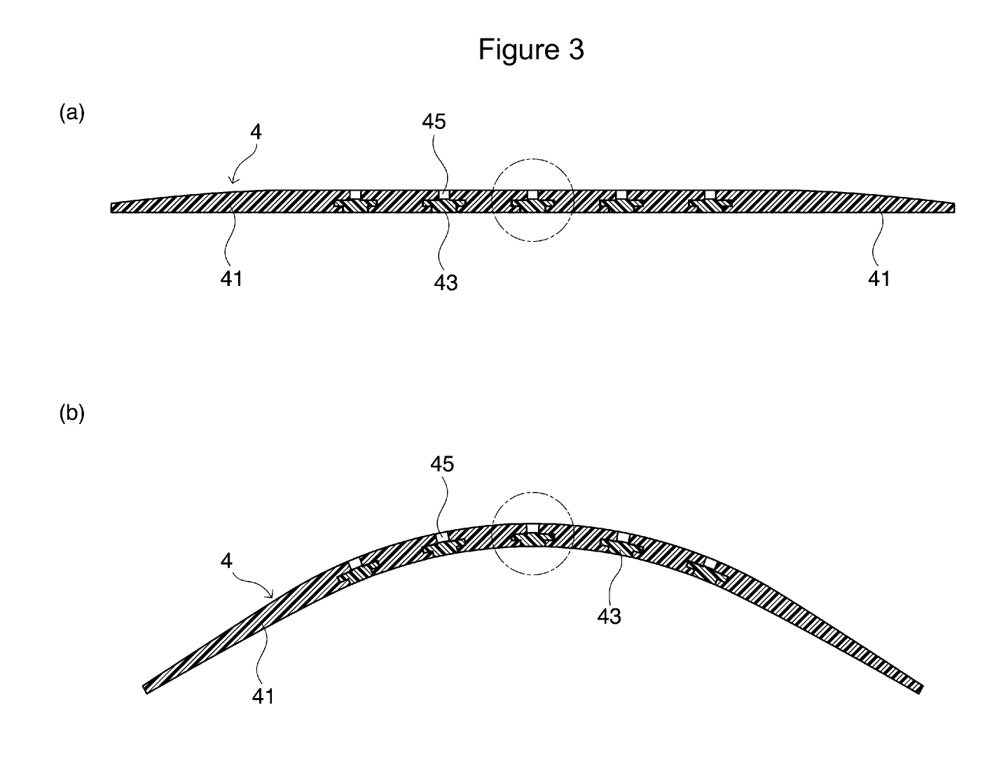

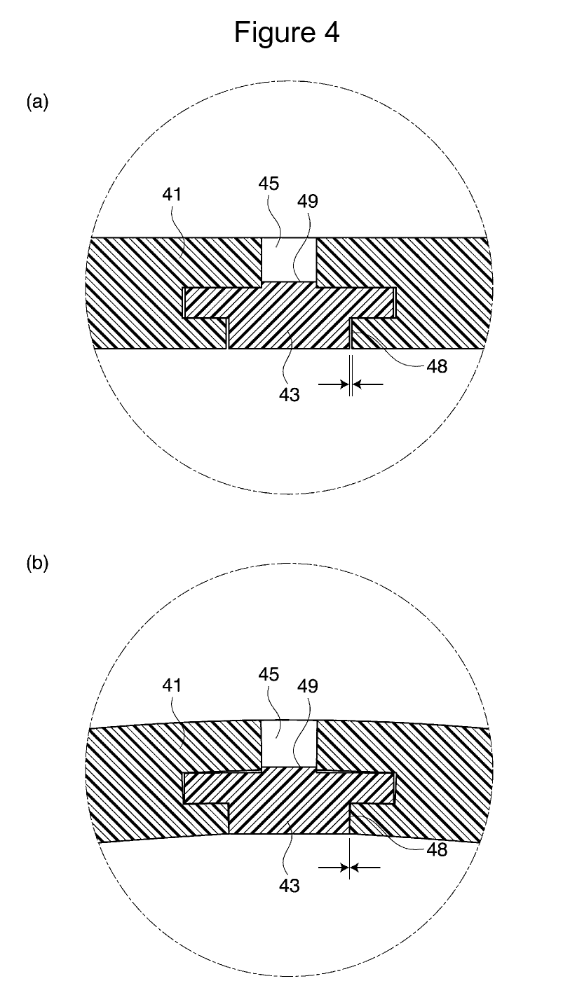

The supporter 4 is substantially in a linear state in the normal state, as shown in FIG. 3(a). At this time, as clearly shown in the enlarged view of FIG. 4(a), gaps 48 are formed between the base 41 and the filler 43. Because five fillers are used in this example, there are ten gaps 48 (five gaps×two (two sides)). However, if a weight is applied to the supporter 4, it bends as shown in FIG. 3(b).

At this time, as clearly shown in the enlarged view of FIG. 4(b), the gaps 48 are closed. As a result, because the supporter is not bent in a predetermined curvature or greater, excessive bending can be suppressed.

This supporter can be used for various purposes as described above.

### Use Example 1

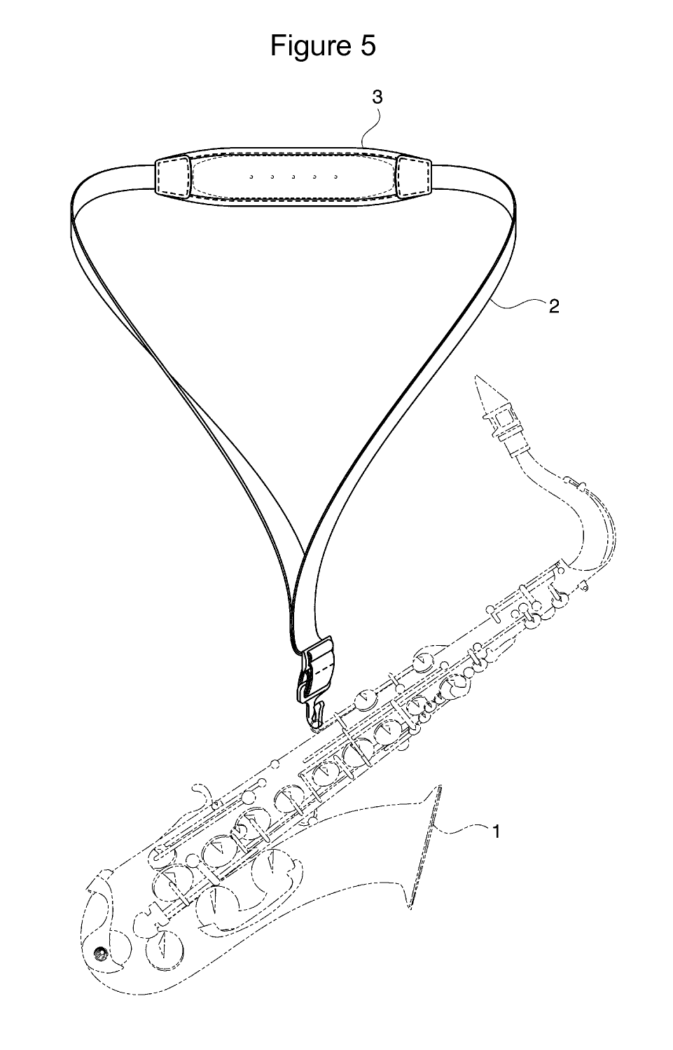

FIG. 5 shows a use example as a strap to which a supporter for a neck guard is attached. A saxophone 1 is worn on the neck of a musician (not shown) via a strap 2. A neck guard 3 is provided in an area of the strap 2 that is placed on the neck

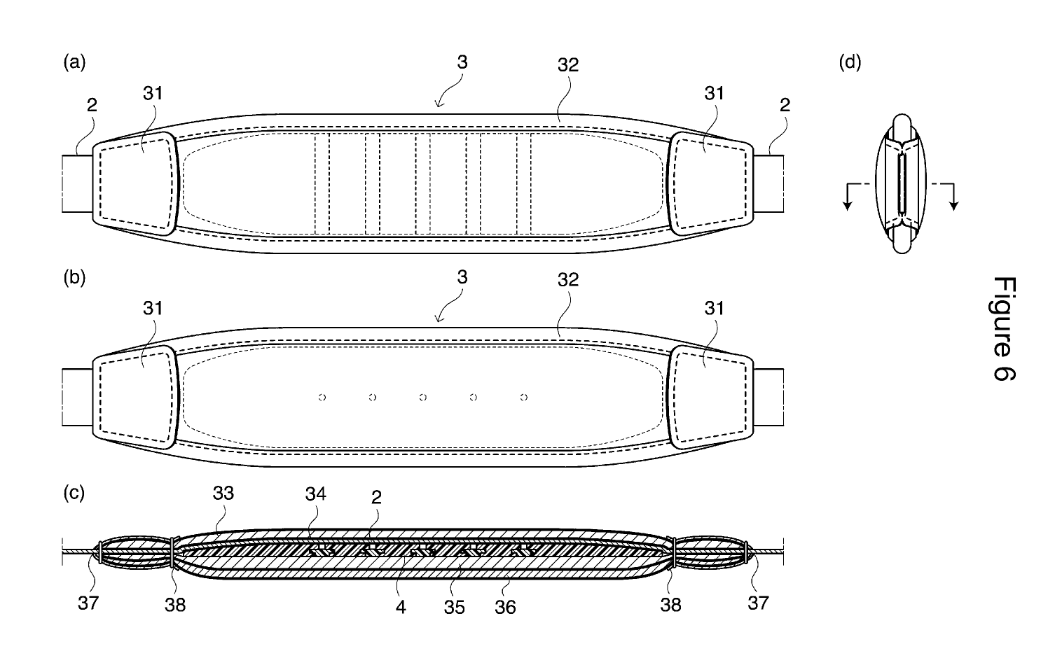

As shown in FIG. 6, the strap 2 passes through and is attached to the neck guard 3, and thus the neck guard 3 is movable along the strap 2.

This neck guard 3 includes right and left end portions 31 and a central portion 32 that constitutes a major area of the neck guard. The central portion 32 includes, from the upper side (outer side of the neck guard, from the wearer's perspective), a surface member 33, a first cushion layer 34, the strap 2, the supporter 4, a second cushion layer 35, and a surface member 36. It is preferable that the surface member 33 is made of a durable material such as a natural leather, an artificial leather, and the like in terms of the durability and appearance. It is preferable that the first and second cushion layers 34 and 35 are made of a soft foamed resin such as polyurethane, polyvinyl chloride, and the like. It should be noted that the feature of the present invention is the above-described supporter 4, and the number of cushion layers and the material are arbitrarily determined.

The main purpose of the right and left end portions 31 is for providing an opening 37 for the strap 2, and for fixing the position of the supporter 4 at the central portion 32. The configuration thereof is the same as that of the central portion 32, except that the supporter 4 is not included. Spaces between the right and left end portions 31 and the central portion 32 are closed using a string 38, and the position of the supporter 4 is fixed.

If the strap for a neck guard in which the supporter of the present invention is utilized is used, the strap is unlikely to dig into the neck and pain is reduced even if the musician plays the saxophone for a long period of time. As a result, it has been verified that the player can breathe significantly easily while he or she is playing a musical instrument

### Use Example 2

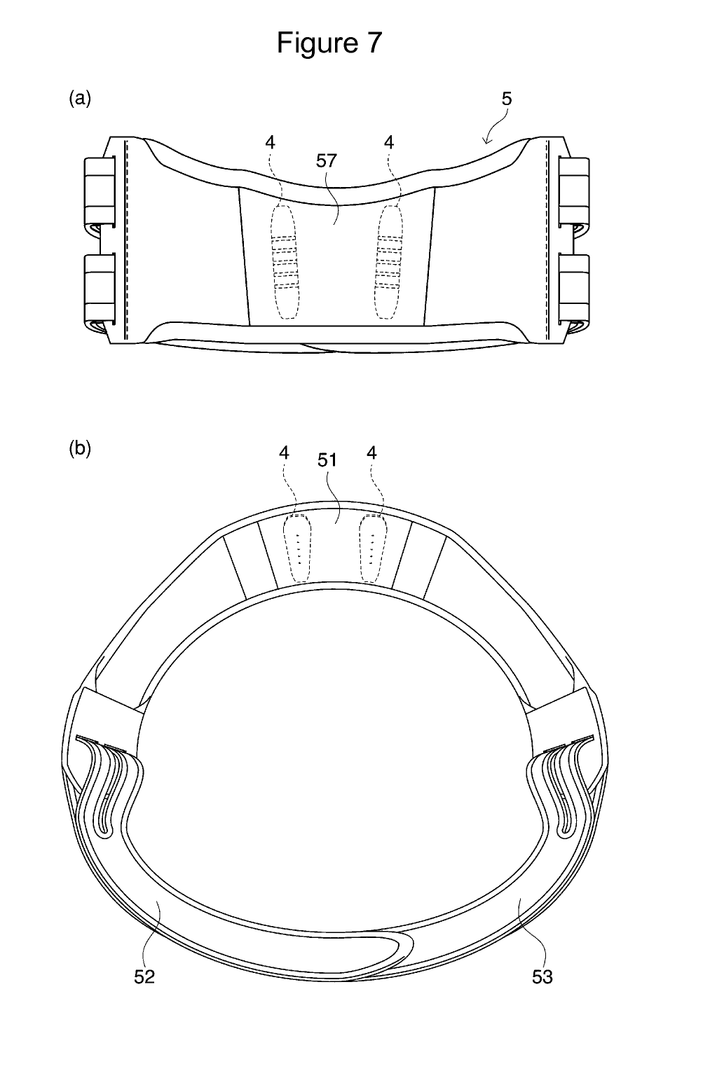

FIG. 7 shows a use example as a lumbar region belt 5 to which a supporter is attached.

This lumbar region belt 5 includes a wide-width central portion 51 that is to be wrapped around the lumbar region or the gluteal region, tip portions 52 and 53 that respectively extend from the wide-width central portion 51 toward left and right, a touch fastener (not shown) of one tip portion, and a touch fastener attachment portion of the other tip portion. Two supporters 4 are attached into the cloth of a back portion of the lumbar region belt 5 in the vertical direction.

If the lumbar region belt 5 is used, the lumbar region is stably supported while maintaining the degree of freedom to some extent

### Use Example 3

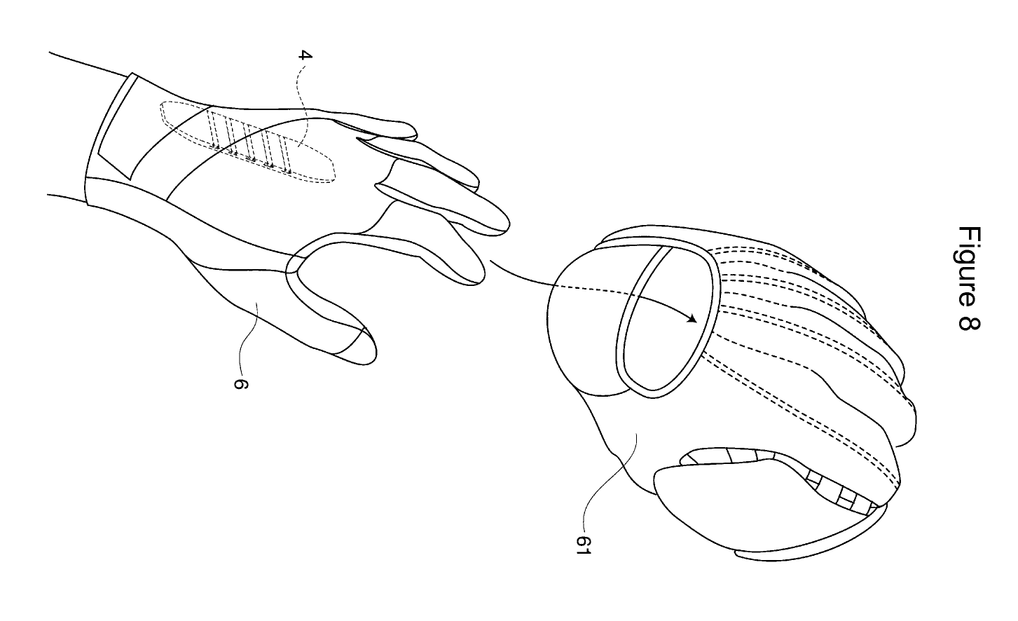

FIG. 8 shows a use example as a glove 6 to which a supporter is attached. One supporter 4 is attached into the cloth on the back side of a hand of a user. If the user wears a catcher's mitt 61 and catches a ball in this state, for example, even if the pitcher throws the ball very fast, it is possible to prevent the wrist of the user from being bent abnormally.

### Use Example 4

Although not shown, it is possible to protect ankles using socks to which these supporters are attached. Also, similarly, it is possible to protect a joint using a joint protector to which this supporter is attached.

### DESCRIPTIONS OF REFERENCE NUMERALS

1 Saxophone
2 Strap
3 Neck guard
31 Right and left end portions
32 Central portion
33 Surface member
34 Cushion layer
35 Cushion layer
36 Surface member
37 Opening
38 String
4 Supporter
41 Base
42 Groove
43 Filler
45 Through hole
46 Base portion
47 Narrow-width portion
48 Gap
49 Small protrusion
5 Lumbar region belt
51 Wide-width central portion
52 Tip portion
6 Glove
61 Catcher's mitt

1. A supporter comprising:
a base (41);
a plurality of grooves (42) that are provided in a width direction of the base (41);
fillers (43) that can be respectively inserted into the plurality of grooves (42); and
disconnection preventing lips (44) for the fillers (43).

2. The supporter according to claim 1, wherein
the fillers (43) each have a small protrusion (49) that extends downward, the base (41) has a through hole (45) or a depression in each of the grooves (42), and the small protrusion (49) and the through hole (45) can be fitted to each other and freely move to such an extent that they do not separate from each other.

3. The supporter according to claim 1, wherein
the disconnection preventing lips (44) are extending portions that each extend from two sides of an upper end of a groove (42) toward the groove.

4. The supporter according to claim 1, wherein
the fillers (43) each have a shape complementary to the grooves (42), and can be inserted therein in a sliding manner in a lateral direction.

5. A strap for a neck guard, the strap comprising the supporter according to claim 1.

6. A lumbar region belt (5), comprising the supporter according to claim 1.

7. A glove (6), comprising the supporter according to claim 1.

8. A sock, comprising the supporter according to claim 1.

9. A joint protector, comprising the supporter according to claim 1.

10. The supporter according to claim 2, wherein
the disconnection preventing lips (44) are extending portions that each extend from two sides of an upper end of a groove (42) toward the groove.

11. The supporter according to claim 2, wherein
the fillers (43) each have a shape complementary to the grooves (42), and can be inserted therein in a sliding manner in a lateral direction.

12. The supporter according to claim 3, wherein
the fillers (43) each have a shape complementary to the grooves (42), and can be inserted therein in a sliding manner in a lateral direction.

13. A strap for a neck guard, the strap comprising the supporter according to claim 2.

14. A lumbar region belt (5), comprising the supporter according to claim 2.

15. A glove (6), comprising the supporter according to claim 2.

16. A glove (6), comprising the supporter according to claim 3.

17. A sock, comprising the supporter according to claim 2.

18. A sock, comprising the supporter according to claim 3.

19. A joint protector, comprising the supporter according to claim 2.

20. A joint protector, comprising the supporter according to claim 3.
